(19) United States
(12) Patent Application Publication
(10) Pub. No.: US 2003/0053963 A1
(43) Pub. Date: Mar. 20, 2003

(54) Compositions and methods relating to dry dental fluoride rinses

(75) Inventors: Joseph J. Pellicano, Sammamish, WA (US); David E. Watton, Kirkland, WA (US)

Correspondence Address: Joshua King, GRAYBEAL JACKSON HALEY LLP, Suite 350, 155 - 108th Avenue, NE, Bellevue, WA, 98004-5973, US

(73) Assignee: Pascal Company, Inc., Bellevue, WA

(21) Appl. No.: 10/176,165

(22) Filed: May 10, 2002

Related U.S. Application Data
(63) Continuation of application No. 09/760,252, filed on Jan. 12, 2001, now abandoned.

(51) Int. Cl. A61K 7/18

(52) U.S. Cl. 424/52

(57) ABSTRACT

A dental fluoride rinse system comprising two different dental rinse compositions, typically maintained in separate vessels until the time that they are mixed with an appropriate solvent such as water. The first dental composition provides a non-stannous fluoride ion source and a phosphate ion source that has a pH of about 3.75+ or −0.75 when mixed with water and comprises greater than about 1% by weight of the fluoride ion. The second dry composition provides between about 0.1% and 1% stannous fluoride when mixed with the solvent and is administered to the patient after the first rinse composition so that the patient gets two different kinds of fluoride administered to the oral cavity in a short time period. In a significant advantage of the present system, the stannous fluoride can be mixed with water by the user.

### FIELD OF THE INVENTION

The field of the present invention is dental rinses.

### BACKGROUND OF THE INVENTION

Teeth are afflicted by several problems including cavities, or caries, and erosion of the enamel, for example by chemical dissolution. These problems can be major health issues for children as well as adults. Additionally, lack of good dental health can be an increasingly severe problem for aging adults that still have their own teeth, for example due to failing motor coordination, reduced salivary secretion, lack of motivation to carry out the proper oral hygiene and dental care, the use of medication or just general ill health.

The primary component of the enamel and dentin in teeth is calcium phosphate in the form of calcium hydroxyapatite. At typical oral pHs, this material is generally insoluble. However, cavities form in the teeth for example when the teeth are subjected to acids produced from the glycolysis of sugars by oral bacteria.

Put simply, the cavities and erosion represent a change in the equilibrium state between the demineralization and remineralization of the enamel and dentin such that demineralization or erosion takes place. The oral remineralizing capacity of the mouth is typically maintained by the saliva, particularly the calcium and phosphate contained in the saliva and the buffering capacity of the saliva. Saliva is super-saturated with respect to the calcium phosphate ions. Therefore, calcium helps protect the teeth against the demineralization and can slowly remineralize the teeth which have become demineralized by acids. U.S. Pat. Nos. 6,054,119; 6,036,944; 4,071,615; and, 4,716,035.

It is well known that fluoride ions can enhance the natural remineralization process. Accordingly, one of the accepted mechanisms by which fluoride toothpaste and rinses protect against caries is the provision of such fluoride ions. The importance of fluoride used in dental care lies in the enhancement of remineralization. Even small repeated fluoride concentrations in the mouth are advantageous. This is because the apatite (i.e., calcium) crystals deposited on the hard tissues of the tooth are more resistant to acids and more irregular in structure than those formed without fluoride.

Dental rinses that provide fluoride and other helpful substances to the oral cavity can be used either at home or in a dental office. However, the transportation and storage of such rinses can be highly problematic because the rinses are typically primarily made up of a large bulk-providing liquid solvent such as water or glycerin, which makes the rinses difficult and expensive to transport and store in large quantities for both size and economic reasons. In addition, rinses comprising stannous fluoride can be particularly difficult because they require the use of a totally anhydrous state because the stannous fluoride is not stable over extended periods of time in the presence of water.

Accordingly, there has gone unmet a need for simple, inexpensive provision of dental rinses particularly dental rinses comprising stannous fluoride that can be useful in a water solvent, or the provision of other fluoride ion sources. The present invention provides these and other advantages.

### SUMMARY OF THE INVENTION

The present invention provides a dental fluoride rinse system comprising two different, dry dental rinse compositions. When maintained in a dry state, the two different compositions are maintained in separate vessels until the time that they are mixed with an appropriate solvent, typically water, at the office of a dentist or other dental care provider, or in the home. The first dental composition provides a fluoride ion source other than stannous fluoride and a phosphate ion source in mixture with a pharmaceutically acceptable carrier or diluent such that when the composition is mixed with water at the dental office the pH of the resulting solution is about 3.75+ or −0.75 and comprises greater than about 1% by weight of the fluoride ion. The second composition provides stannous fluoride that is used by the patient as a follow up rinse to the initial composition so that the patient gets two different kinds of fluoride administered to the oral cavity in a short time period. Because the stannous fluoride is provided to the user in a dry form, it can be mixed with water by the user. This is a significant advantage over previous liquid systems, where an anhydrous solvent such as glycerin was used. This promotes ease of use and administration for the patient, and lowers the costs associated with the shipping, storage and production of the ultimate dental rinse product.

In one aspect, the present invention provides a dental fluoride rinse system comprising a) a first dry dental rinse composition comprising an acidifier, a fluoride ion source other than stannous fluoride able to provide a fluoride ion when mixed with water, an effective amount of a phosphate ion source, and a pharmaceutically acceptable carrier or diluent, wherein when the first dry dental rinse composition is mixed with a desired amount of water the resulting solution is about pH 3.75±0.75 and comprises greater than about 1% by weight of the fluoride ion; and, b) a second dry dental rinse composition comprising stannous fluoride and a pharmaceutically acceptable carrier or diluent.

In some embodiments, when the second dry dental rinse composition is mixed with a desired amount of water the resulting solution comprises between about 0.1% and 1% stannous fluoride. The first and second compositions can further comprise a desired flavoring, a sweetener or a buffer, and the acidifier of the first rinse composition comprises an acidic buffer.

The fluoride ion source of the first rinse composition can be selected from the group consisting of sodium fluoride, lithium fluoride, sodium monofluorophosphate, salts of fluorosilic acid, sodium fluoborate, sodium bifluoride, potassium bifluoride, strontium fluoride, indium fluoride, zirconium fluoride, ammonium fluoride, amine fluoride, titanium tetra fluoride, sodium hexafluorostannate, sodium fluorozirconate, potassium fluorozirconate, ammonium fluorozirconate, copper fluoride, nickel fluoride, palladium fluoride, manganese fluoride, stannous chlorofluoride and stannous fluorozirconate. The fluoride ion source can be an alkali metal fluoride selected from the group consisting of sodium fluoride, potassium fluoride and mixtures thereof. The acidifier can be selected from the group consisting of citric acid, lactic acid, malic acid, and tartaric acid. The pharmaceutically acceptable carrier or diluent can be selected from the group consisting of sorbitol, xylitol, tartaric acid, salts of citric acid, salts of lactic acid, block copolymers of ethylene oxide and propylene oxide nonionic surfactants, sodium acetate, sodium bicarbonate and sodium chloride. The phosphate ion source can be selected from the group consisting of monobasic sodium phosphate, dibasic sodium phosphate, tribasic sodium phosphate, sodium pyrophosphate, and potassium pyrophosphate.

The first rinse composition and the second rinse composition can be contained in separate vessels, such as foil pouches. The dental fluoride rinse system can further comprise a graduated mixing cup sized to receive and measure a predetermined amount of a pharmaceutically acceptable solvent between about 10 ml and 20 ml and to permit mixing of at least one of the first and second rinse compositions with the solvent. The first rinse composition and the second rinse composition are powders.

In another aspect, the present invention provides a dental fluoride rinse system comprising: a) a first dry dental rinse composition comprising a means for acidifying, a means for providing a fluoride ion other than stannous fluoride, means for providing a phosphate ion, a pharmaceutically acceptable carrier or diluent means, and a buffer, wherein when the first dry dental rinse composition is mixed with water the resulting solution is about pH 3.75±0.75 and comprises greater than about 1% by weight of the fluoride ion; and, b) a second dry dental rinse composition comprising a means for providing a stannous fluoride and a pharmaceutically acceptable carrier or diluent means, wherein when the second dry dental rinse composition is mixed with a desired amount of water the resulting solution comprises between about 0.1% and 1% stannous fluoride. The first and second compositions can further comprise at least one of a means for providing a desired flavor, a buffer means, and a means for providing artificial sweetening.

In a further aspect, the present invention provides a method of preparing a dental fluoride rinse comprising: a) mixing a first solvent with a first dry dental rinse composition comprising an acidifier, a fluoride ion source other than stannous fluoride able to provide fluoride ion when mixed with water, a phosphate ion source and a pharmaceutically acceptable carrier or diluent, to provide a first dental rinse of about pH 3.75±0.75 and comprising greater than about 1% by weight of the fluoride ion; and b) mixing a second solvent with a second dry dental rinse composition comprising stannous fluoride and a pharmaceutically acceptable carrier or diluent to provide a second dental rinse comprising between about 0.1% and 1% stannous fluoride.

In some embodiments, the second solvent comprises water, or the first and second dry dental rinse compositions each further comprise at least one of a desired flavoring and an artificial sweetener.

In still another aspect, the present invention provides a method of administering a dental fluoride rinse to a patient comprising: a) mixing water with a first dry dental rinse composition comprising an acidifier, a fluoride ion source other than stannous fluoride able to provide fluoride ion when mixed with water, a phosphate ion source and a pharmaceutically acceptable carrier or diluent, to provide a first dental rinse of about pH 3.75±0.75 and comprising greater than about 1% by weight of the fluoride ion; b) mixing water with a second dry dental rinse composition comprising stannous fluoride and a pharmaceutically acceptable carrier or diluent to provide a second dental rinse comprising between about 0.1% and 1% stannous fluoride; and, c) serially administering the first dental rinse and then the second dental rinse to a patient.

In some embodiments, the first and second dry dental rinse compositions a administered to a patient within about 20 minutes of the mixing. The first and second dry dental rinse compositions can each further comprise at least one of a desired flavoring and an artificial sweetener.

In still yet another aspect, the present invention provides a kit comprising a first vessel containing a first dry dental rinse composition as described herein and a second vessel containing a second dry dental rinse composition as described herein. The first and second vessels can be foil pouches. The kit can comprise a first means for containing a first dry dental rinse composition means and a second means for containing a second dry dental rinse composition means.

These and other aspects, features and embodiments of the present invention are set forth within this application, including the following Detailed Description. In addition, various references are set forth herein that describe in more detail certain compositions, apparatus, methods and other information; all such references are incorporated herein by reference in their entirety and for all their teachings and disclosures, regardless of where they may appear in this application.

### DETAILED DESCRIPTION OF THE INVENTION

The present invention provides a dental fluoride rinse system. The system comprises two different dental rinse compositions maintained in separate vessels until the time that they are mixed with an appropriate solvent such as water. The first dental composition provides a non-stannous fluoride ion source and a phosphate ion source that has a pH of about 3.75+ or −0.75 and comprises greater than about 1% by weight of the fluoride ion when mixed with water. The second dry composition provides stannous fluoride and is used in combination with the initial composition so that the patient gets two different kinds of fluoride administered to the oral cavity in a short time period. In a significant advantage of the present system, the stannous fluoride can be mixed with water by the user.

Definitions:

The following paragraphs provide definitions of some of the terms used herein. All terms used herein, including those specifically described below in this section, are used in accordance with their ordinary meanings unless the context or definition indicates otherwise. Also unless indicated otherwise, except within the claims, the use of “or” includes “and” and vice-versa. Non-limiting terms are not to be construed as limiting unless expressly stated (for example, “including” means “including without limitation” unless expressly stated otherwise).

“Composition” indicates a combination of multiple substances into an aggregate mixture.

Pharmaceutically acceptable carriers or diluents are non-toxic to recipients at the dosages and the concentrations employed. The carrier or diluent is a solid carrier or diluent suitable for enhancing the administration of the fluoride ion and other materials in the compositions of the present invention. The carriers or diluents are compatible with dry compositions, whether in powder, granule, tablet or otherwise. Where the compositions of the present invention comprise aggregations such as granules or tablets, the compositions can additionally further comprise a binder, which is a substance that produces or promotes cohesion in loosely assembled substances. A single substance can function as one or more of a carrier, diluent, or binder provided that it has each of the respective properties.

The terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

Other terms and phrases in this application are defined in accordance with the above definitions, and in other portions of this application.

Turning to a more detailed discussion of the invention, in a first aspect the present invention provides a dental fluoride rinse system comprising two dry compositions. The first composition is a first dry dental rinse composition, which means that the composition is not a liquid, but is, rather, a dry substance such as a powder, granule or tablet. In certain alternative embodiments, the dental fluoride rinse compositions can be an anhydrous gel.

The first dry dental rinse composition comprises an acidifier which means a substance capable of rendering the composition acidic when it is mixed with water. In preferred embodiments, the acidifier is citric acid, tartaric acid, malic acid, lactic acid, and acidic phosphate buffer, sodium pyrophosphate, or potassium pyrophosphate.

The first dry dental rinse composition also comprises a fluoride ion source other than stannous fluoride, while the second dried dental rinse comprises a stannous fluoride. The fluoride ion source is able to provide fluoride ion when mixed with water. The first dried dental rinse composition can additionally comprise stannous fluoride if desired. The fluoride ions are included in the compositions in an effective amount, which means an amount adequate to significantly enhance the remineralization of enamel or dentin. Generally, the dry compositions comprise from about 1% to about 35% by weight of the fluoride ion source, preferably from about 10% to about 30% by weight, and more preferably from about 15% to about 25% by weight, as well as other percentages within the broadest of such ranges, to give greater than about 1% by weight of the fluoride ion when diluted with water, preferably from about 1% to about 2% by weight, and more preferably from about 1.2% to about 1.4% by weight. The fluoride ion in the first composition can also be known as APF, or acidulated phosphate fluoride.

The second dry dental rinse of the present invention comprises an effective amount of stannous fluoride. As above, an effective amount of the stannous fluoride means an adequate amount of stannous fluoride to significantly enhance the remineralization of the enamel or dentin of the teeth. In certain preferred embodiments, the stannous fluoride is present in the dry composition from between 1% to 30%, preferably from about 4% to 20%, and further preferably from about 6% to 12% by weight, as well as other percentages within the broadest of such ranges, to give about 0.01% to about 1% by weight of the stannous fluoride when diluted with water, preferably from about 0.1% to about 1% by weight, and more preferably from about 0.3% to about 0.7% by weight.

Fluoride ion sources include sodium fluoride, lithium fluoride, sodium monofluorophosphate, salts of fluorosilic acid, sodium fluoroborate, sodium bifluoride, potassium bifluoride, strontium fluoride, indium fluoride, zirconium fluoride, ammonium fluoride, amine fluoride, titanium tetra fluoride, sodium hexafluorostannate, sodium fluorozirconate, potassium fluorozirconate, ammonium fluorozirconate, copper fluoride, nickel fluoride, palladium fluoride, manganese fluoride, stannous fluoride, stannous chorofluoride and stannous fluorozirconate.

At least the first dental rinse composition additionally comprises a phosphate ion source, which is preferably an alkaline metal phosphate ion source, such as mono-basic, dibasic, or tri-basic sodium phosphate, sodium pyrophosphate, potassium pyrophosphate and lithium phosphate. Additional suitable phosphate ion sources include alkaline metal polyphosphate and hydrogen phosphate. It is possible to provide both phosphate and fluoride ion in a single substance, for example, in the compound sodium monofluorophosphate. The phosphate ion sources can provide a buffering function or an acidifying function, if desired.

The pharmaceutically acceptable carrier, diluent, and binder, as noted above, is non-toxic to patients at the dosage and concentrations employed. Preferred embodiments include sorbitol, xylitol, tartaric acid, salts of citric acid, salts of lactic acid, block copolymers of ethylene oxide and propylene oxide nonionic surfactants (available from BASF Corporation, Mount Olive, N.J.), sodium acetate, sodium bicarbonate and sodium chloride. In preferred embodiments, the pharmaceutically acceptable carrier or diluent is sorbitol, xylitol, or mixtures thereof. The pharmaceutically acceptable carrier, diluent, or binder is preferably added in an amount such that accurate weighing or other measuring of the effective or active ingredients within the compositions is facilitated.

In some embodiments, the dry compositions of the present invention further comprise a desired flavoring typically from about 0.1% to about 3% by weight of the composition, preferably from about 1% to about 2%. The flavorings can be artificial or natural flavors such as spearmint, peppermint, wintergreen, dried essential oils, aldehydes, esters, alcohols, or similar materials, sassafras, clove, sage, eucalyptus, cinnamon, lemon-lime, grapefruit, orange, or menthol.

The compositions can also comprise a buffer to assist in the control of the pH of the composition. In preferred embodiments, the buffer can be an acidic buffer, which means that the buffer itself establishes the pH of the composition once it is mixed with water. Buffers were discussed above, and as with other constituents of the compositions of the present invention, suitable alternatives will be known to a person of ordinary skill in life in the art in view of the present disclosure.

The present invention can additionally comprise an artificial sweetener such as saccharin, dextrose, levulose, xylitol, sorbitol, sodium cyclamate, and aspartame, typically from about 0.1% to about 30.0% by weight of the compositions, preferably from about 1% to about 20%.

The compositions of the present invention are contained in separate vessels, for example for shipping and storage. The vessels can be any vessels known in the art and are preferably airtight and watertight. Further preferably, the containers add little to the overall weight and bulk of the compositions. The containers can be made of foil, plastic, glass, or any other desirable substance. In a preferred embodiment, the vessels are foil pouches, wherein each foil pouch contains a single “serving” of the compositions, e.g., a first packet contains about 1 gram of the first dry dental rinse composition comprising sodium fluoride or other similar fluoride ion source, and a second packet contains about 1 gram of the second dry dental rinse comprising stannous fluoride. In a further preferred embodiment, the two vessels containing the different dry dental rinse compositions are color coded or otherwise plainly labeled such that it is easy to see which of the two compositions is contained therein.

In an additional embodiment, the dental fluoride rinse systems of the claimed invention can further comprise a graduated mixing cup that is sized to receive and measure the compositions of the invention and a predetermined amount, such as 5 ml, of a pharmaceutically acceptable solvent. It is preferred that the solvent for use with the first dry rinse composition comprising sodium fluoride or other similar fluoride ion source be water or a pharmaceutically acceptable solvent having properties similar to water. Regarding the second dry dental rinse composition, the pharmaceutically acceptable solvent can be glycerin or other non-hydrous solvent, but it is also a feature of the present invention that, because the second dry dental rinse composition will be maintained in the pharmaceutically acceptable solvent for a relatively short period of time (typically 5 seconds to 10 minutes), before introduction to the oral cavity, the stannous fluoride-containing compositions of the present composition can be mixed with water or other hydrous solvent. This feature significantly simplifies the implementation and use of the stannous fluoride-containing compositions, both at home and in a dental office. A graduated mixing cup is used herein its normal sense to indicate a cup, glass, or other drinking vessel that has indications, typically on the side, that a certain fill level has been obtained.

In additional aspects, the present invention provides methods of making, preparing, and using the compositions of the present invention. The compositions can be made using traditional methods of mixing the various substances required for the compositions, as well as additional, optional substances that may be desired such as sweeteners, flavorings, buffers, and acidic buffers. The mixing of the compositions of the claimed invention with water is also performed in a traditional sense, except that it is highly advantageous, as noted above, that the compositions comprising stannous fluoride can be mixed with water at the location where the patient will be using the compositions, and therefore, the deleterious effects of having stannous fluoride in contact with a hydrous solution are minimized, and potentially even eliminated, despite the fact that a hydrous solution can be used.

The first dental rinse and the second dental rinse are administered to the patient serially. For example, the first dried dental rinse composition and the second dried dental rinse composition can be mixed separately with a solvent(s), and the resulting first dental rinse is administered to a patient immediately before administration of the second dental rinse to the patient, although a relatively brief interval between administration of the two compositions is acceptable. Typically, the interval between the two administrations is from about 5 seconds to about 30 minutes, preferably from about 30 seconds to 5 minutes. This administration of one dental rinse before the other can be referred to as serially administering the dental rinses.

### EXAMPLES

### Example 1

### Sound Enamel Solubility Reduction Study

Purpose:

The purpose of this in vitro study was to determine the ability of fluoridated topical treatments to prevent demineralization of sound enamel. The test procedure was similar to Procedure 33 in the FDA Monograph for dentifricers except that it used sound enamel rather than incipient lesion enamel.

Procedure:

Sound bovine enamel specimens (10×10 mm) were prepared from incisors. They were mounted in methylmethacrylate block designed to fit the prophy apparatus. The surfaces were ground and polished smooth to remove any outer fluoride layer. Twelve specimens per group were prepared and mounted on the end of plastic rods.

To determine baseline solubility, each specimen was etched in 10 ml of 0.1M lactic acid buffer for 15 minutes with constant stirring. A sample of that etch solution (2.5 ml) was then diluted to 25 ml with the appropriate reagents to form the color change and the amount of phosphorus determined.

The specimens were then randomized into groups and immersed into the appropriate topical test as indicated below in the chart describing the test agents. All treatments were 37° C. (preheat teeth). The test agent was then rinsed from the teeth for 30 seconds following the individual tooth treatment. All two-part treatments were performed within 10 minutes of mixing.

To remove loosely bound CaF, the specimens were immersed in 1.0 N KOH saturated with calcium phosphate tribasic for 18 hours under constant agitation. Following KOH immersion, the specimens were rinsed with DI (deionized) water. The specimens were then etched as described above and the post-treatment phosphorus release determined.

Calculation of Enamel Solubility Reduction:

The percent of enamel solubility reduction (“ESR”)was computed as the difference between the amount of phosphorus in the pre- and post-treatment decalcification solutions, divided by the amount of phosphorus in the pre-treatment solution and multiplied by 100.

Statistical Analyses:

Statistical analyses were performed with a one-way analysis of variance model of the Sigma Stat Statistical Software (2.0). Since the ANOVA indicated significant differences, the individual means were analyzed by the Student Newman-Keuls (SNK) test.

Test Agents:
The test agents were as follows:

| Group | Topical Agent | Time | Mixture |
|---|---|---|---|
| 1 | Placebo/control | 1 minute | NA |
| | (DI Water) | | |
| 2 | Puff ® APF Foam* | 1 minute | NA |
| 3 | 60 Second Taste ® | 1 minute | NA |
| | APF Gel** | | |
| 4 | Two-part liquid rinse | 2 minutes | 4 parts A + 1 part B |
| | administered together | | |
| 5 | Two-part rinse from | 2 minutes | 1 part A + 1 part B |
| | powder administered | | +37 parts water |
| | together | | |
| 6 | Two-part rinse from | 1 minute + | 1 part A + 15 parts |
| | powder administered | 1 minute | water then 1 part B |
| | sequentially | | then 15 parts water |

*1.23% fluoride ion in a foam carrier, approximate pH 3.75, no stannous fluoride.
**1.23% fluoride ion in a gel carrier, approximate pH 3.5, no stannous fluoride.

Results:

In summary, all the fluoride-containing agents promoted a significant reduction in enamel solubility compared to the control. The two-part systems were more effective that the APF gel which was more effective than the Puff APF foam. All three of the two-part systems were significantly more effective than the gel or foam, although the treatment time was twice as long.

TABLE 1
Summary

| | Pre-Etch | | | Post-Etch | | Delta | | Percent | |
|---|---|---|---|---|---|---|---|---|---|
| Treatment | μP | | | μP | | μP | | Reduction | |
| DI Water | 530 ± 43* | | ** | 646 ± 39 | | −116 ± 17 | | −24.7 ± 4.5 | |
| Puff ® APF Foam | 563 ± 41 | | | 389 ± 25 | | 174 ± 34 | | 30.0 ± 4.0 | |
| 60 Second Taste ® APF Gel | 598 ± 29 | | | 242 ± 8 | | 356 ± 25 | | 58.9 ± 1.6 | |
| Two-Part Powder - Simultaneous | 533 ± 40 | {close oversize bracket} | | 145 ± 12 | | 388 ± 33 | | 72.2 ± 1.8 | |
| Two-Part Liquid - Simultaneous | 547 ± 41 | | | 116 ± 8 | {close oversize bracket} | 431 ± 39 | {close oversize bracket} | 77.8 ± 2.1 | {close oversize bracket} |
| Two-Part Powder - Sequential | 513 ± 24 | | | 102 ± 5 | | 411 ± 24 | | 79.8 ± 1.2 | |

*Mean ± SEM (N = 12)
**Values within brackets do not differ significantly (p > 0.05) relative to each other as determined by Student Newman-Keuls analysis.

TABLE 2
Group 1: Deionized Water (Control)
Phosphorus Data (μg Found)

| TOOTH SET | PRE | POST | DELTA | % RED. |
|---|---|---|---|---|
| 1 | 453 | 661 | −207 | −45.69 |
| 2 | 666 | 793 | −127 | −19.15 |
| 3 | 496 | 650 | −154 | −31.06 |
| 4 | 374 | 586 | −212 | −56.85 |
| 5 | 416 | 501 | −85 | −20.42 |
| 6 | 560 | 602 | −42 | −7.59 |
| 7 | 507 | 655 | −149 | −29.36 |
| 8 | 618 | 751 | −133 | −21.49 |
| 9 | 453 | 528 | −74 | −16.40 |
| 10 | 735 | 767 | −32 | −4.34 |
| 11 | 783 | 862 | −80 | −10.18 |
| 12 | 294 | 395 | −101 | −34.32 |
| MEAN | 530 | 646 | −116 | −24.74 |
| STD. DEV. | 148 | 134 | 58 | 15.60 |
| STD. ERR. | 43 | 39 | 17 | 4.50 |

TABLE 3
Group 2: Puff APF Foam
Phosphorus Data (μg Found)

| TOOTH SET | PRE | POST | DELTA | % RED. |
|---|---|---|---|---|
| 1 | 900 | 395 | 505 | 56.10 |
| 2 | 544 | 368 | 175 | 32.24 |
| 3 | 475 | 368 | 106 | 22.38 |
| 4 | 639 | 512 | 127 | 19.94 |
| 5 | 528 | 448 | 80 | 15.10 |
| 6 | 677 | 432 | 244 | 36.12 |
| 7 | 326 | 151 | 175 | 53.78 |
| 8 | 554 | 406 | 149 | 26.83 |
| 9 | 528 | 363 | 165 | 31.20 |
| 10 | 480 | 432 | 48 | 9.96 |
| 11 | 634 | 437 | 197 | 31.00 |
| 12 | 475 | 352 | 122 | 25.74 |
| MEAN | 563 | 389 | 174 | 30.03 |
| STD. DEV. | 141 | 88 | 117 | 13.83 |
| STD. ERR. | 41 | 25 | 34 | 3.99 |

TABLE 4
Group 3: Taste APF Gel
Phosphorus Data (μg Found)

| TOOTH SET | PRE | POST | DELTA | % RED. |
|---|---|---|---|---|
| 1 | 799 | 262 | 537 | 67.17 |
| 2 | 645 | 283 | 361 | 56.03 |
| 3 | 613 | 262 | 351 | 57.21 |
| 4 | 427 | 214 | 212 | 49.78 |
| 5 | 517 | 225 | 292 | 56.49 |
| 6 | 730 | 267 | 462 | 63.34 |
| 7 | 655 | 225 | 430 | 65.66 |
| 8 | 570 | 188 | 382 | 67.07 |
| 9 | 592 | 262 | 329 | 55.68 |
| 10 | 565 | 241 | 324 | 57.35 |
| 11 | 565 | 241 | 324 | 57.35 |
| 12 | 501 | 230 | 271 | 54.05 |
| MEAN | 598 | 242 | 356 | 58.93 |
| STD. DEV. | 101 | 27 | 88 | 5.55 |
| STD. ERR. | 29 | 8 | 25 | 1.60 |

TABLE 5
Group 4: Two Part Rinse From Liquid
Phosphorus Data (μg Found)

| TOOTH SET | PRE | POST | DELTA | % RED. |
|---|---|---|---|---|
| 1 | 671 | 124 | 547 | 81.52 |
| 2 | 581 | 108 | 473 | 81.39 |
| 3 | 522 | 44 | 478 | 91.50 |
| 4 | 746 | 119 | 627 | 84.07 |
| 5 | 522 | 119 | 404 | 77.27 |
| 6 | 352 | 135 | 218 | 61.79 |
| 7 | 384 | 108 | 276 | 71.87 |
| 8 | 565 | 119 | 446 | 78.98 |
| 9 | 379 | 103 | 276 | 72.87 |
| 10 | 416 | 103 | 313 | 75.30 |
| 11 | 655 | 151 | 505 | 77.01 |
| 12 | 767 | 156 | 611 | 79.66 |
| MEAN | 547 | 116 | 431 | 77.77 |
| STD. DEV. | 144 | 28 | 135 | 7.27 |
| STD. ERR. | 41 | 8 | 39 | 2.10 |

TABLE 6
Group 5: Two Part Rinse From Powder - Administered Simultaneously
Phosphorus Data (μg Found)

| TOOTH SET | PRE | POST | DELTA | % RED. |
|---|---|---|---|---|
| 1 | 379 | 135 | 244 | 64.47 |
| 2 | 544 | 167 | 377 | 69.37 |
| 3 | 767 | 172 | 595 | 77.59 |
| 4 | 629 | 129 | 499 | 79.42 |
| 5 | 368 | 119 | 250 | 67.77 |
| 6 | 522 | 103 | 420 | 80.32 |
| 7 | 544 | 156 | 388 | 71.32 |
| 8 | 560 | 124 | 436 | 77.83 |
| 9 | 416 | 98 | 319 | 76.57 |
| 10 | 337 | 124 | 212 | 63.14 |
| 11 | 602 | 167 | 436 | 72.34 |
| 12 | 730 | 246 | 483 | 66.25 |
| MEAN | 533 | 145 | 388 | 72.20 |
| STD. DEV. | 139 | 40 | 115 | 6.06 |
| STD. ERR. | 40 | 12 | 33 | 1.75 |

TABLE 7
Group 6: Two Part Rinse From Powder - Administered Sequentially
Phosphorus Data (μg Found)

| TOOTH SET | PRE | POST | DELTA | % RED. |
|---|---|---|---|---|
| 1 | 661 | 103 | 558 | 84.44 |
| 2 | 480 | 113 | 367 | 76.37 |
| 3 | 475 | 119 | 356 | 74.98 |
| 4 | 491 | 71 | 420 | 85.54 |
| 5 | 581 | 129 | 452 | 77.73 |
| 6 | 639 | 87 | 552 | 86.41 |
| 7 | 517 | 129 | 388 | 74.98 |
| 8 | 496 | 87 | 409 | 82.48 |
| 9 | 507 | 103 | 404 | 79.70 |
| 10 | 437 | 98 | 340 | 77.71 |
| 11 | 517 | 103 | 414 | 80.12 |
| 12 | 352 | 82 | 271 | 76.86 |
| MEAN | 513 | 102 | 411 | 79.78 |
| STD. DEV. | 84 | 19 | 82 | 4.06 |
| STD. ERR. | 24 | 5 | 24 | 1.17 |

### Example 2

### Sound Enamel Fluoride Uptake Study

Purpose:

The purpose of this in vitro study was to determine the effect of topical fluorides on promoting fluoride uptake into sound enamel. The test procedure was similar to the one identified in Procedure 40 in the FDA Monograph for dentifricers except that it used sound enamel rather than incipient lesion enamel.

Procedure:

Sound, upper, central, human incisors were selected and cleaned of all adhering soft tissue. Cores of enamel 3 mm in diameter were prepared from each tooth by cutting perpendicularly to the labial surface with a hollow-core diamond drill bit. This was performed under water to prevent overheating of the specimens. Each specimen was embedded in the end of a plexiglass rod (¼″ diameter×2″ long) using methylmethacrylate. The excess acrylic was cut away exposing the enamel surface. The enamel specimens were polished with 600 grit wet/dry paper and then with micro-fine Gamma Alumina. The resulting specimen was a 3 mm disk of enamel with all but the exposed surface covered with acrylic.

The treatments were performed using the test agents (described below) as provided. The twenty-four specimens were immersed into their assigned test agent with constant agitation for the designated time. Following treatment, the specimens were rinsed with distilled water. They were then immersed in 1.0 N KOH saturated with calcium phosphate tribasic for 18-hours to remove loosely bound calcium fluoride. A constant immersion wheel provided continual agitation. Following the KOH immersion, all the specimens were rinsed with distilled water. One layer of enamel was then removed from each specimen by immersion in 0.5 ml of 1.0 N HClO₄ for 15 seconds. A sample of each solution was then buffered with TISAB at a pH of 5.2 (0.25 ml sample, 0.5 ml TISAB and 0.25 ml 1N NaOH) and the fluoride content determined by comparison to a similarly prepared standard curve (1 ml std and 1 ml TISAB).

Statistical Analyses:

Statistical analyses of the individual means were performed with a one-way analysis of variance model using Sigma Stat (2.0) Software. If the ANOVA indicated significant differences, the individual means were analyzed by the Student Newman-Keuls (SNK) test.

Test Agents:
The test agents were as follows:

| Group | Topical Agent | Time | Mixture |
|---|---|---|---|
| 1 | Placebo/control | 1 minute | NA |
| | (DI Water) | | |
| 2 | Puff ® APF Foam* | 1 minute | NA |
| 3 | 60 Second Taste ® | 1 minute | NA |
| | APF Gel** | | |
| 4 | Two- part liquid rinse | 2 minutes | 4 parts A + 1 part B |
| | administered together | | |
| 5 | Two-part rinse from | 2 minutes | 1 part A + 1 part B |
| | powder administered | | +37 parts water |
| | together | | |
| 6 | Two-part rinse from | 1 minute + | 1 part A + 15 parts |
| | powder administered | 1 minute | water then 1 part B |
| | sequentially | | then 15 parts water |

*1.23% fluoride ion in a foam carrier, approximate pH 3.75, no stannous fluoride.
**1.23% fluoride ion in a gel carrier, approximate pH 3.5, no stannous fluoride.

Results:

In summary, all the fluoride-containing products promoted significant enamel fluoride uptake over the placebo. The two-part rinses that were used for 2 minutes were similar but not as effective as the two APF systems or the two part dry rinse—sequential. The two part dry rinse—sequential (part A for 60 seconds then part B for 60 seconds) was the most effective in promoting enamel fluoride uptake and was significantly more effective than either APF system.

TABLE 8
Summary

| Test Agent | Fluoride (ppm) | | | Thickness (μm) | |
|---|---|---|---|---|---|
| DI Water (control) Placebo | 41 ± 2* | | | 18.9 ± 0.3 | |
| Two Part Powder Simultaneous | 580 ± 29 | | ** | 18.5 ± 0.2 | |
| | | {close oversize bracket} | | | |
| Two Part Liquid Rinse | 636 ± 22 | | | 18.3 ± 0.4 | {close oversize bracket} |
| Taste APF Gel | 1788 ± 101 | | | 15.4 ± 0.3*** | |
| | | {close oversize bracket} | | | |
| Puff APF Foam | 1824 ± 85 | | | 18.5 ± 0.3 | |
| Two Part Powder Sequential | 2651 ± 109 | | | 17.0 ± 0.3 | |

*Mean ± SEM (N = 24)
**Values within brackets do not differ significantly (p > 0.05) as determined by SNK test.
***Value 15.4 is significantly lower than all the others.

TABLE 9
Group 1: Deionized Water - (Control)

| RAW | RAW CAL- | | DEPTH | FLUORIDE |
|---|---|---|---|---|
| FLUORIDE DATA | CIUM DATA | | MIC | PPM |
| 0.017 | 2.55 | | 17.12 | 48.83 |
| 0.016 | 2.90 | | 19.47 | 40.41 |
| 0.012 | 2.73 | | 18.33 | 32.20 |
| 0.016 | 2.67 | | 17.93 | 43.89 |
| 0.014 | 2.90 | | 19.47 | 35.36 |
| 0.011 | 2.60 | | 17.46 | 30.99 |
| 0.017 | 2.97 | | 19.94 | 41.93 |
| 0.020 | 2.77 | | 18.60 | 52.89 |
| 0.019 | 3.09 | | 20.75 | 45.04 |
| 0.016 | 2.78 | | 18.67 | 42.16 |
| 0.013 | 2.74 | | 18.40 | 34.75 |
| 0.017 | 2.75 | | 18.46 | 45.28 |
| 0.016 | 2.60 | | 17.46 | 45.08 |
| 0.013 | 2.24 | | 15.04 | 42.51 |
| 0.020 | 2.62 | | 17.59 | 55.91 |
| 0.012 | 2.70 | | 18.13 | 32.55 |
| 0.013 | 3.01 | | 20.21 | 31.64 |
| 0.017 | 3.04 | | 20.41 | 40.96 |
| 0.012 | 2.95 | | 19.81 | 29.80 |
| 0.014 | 3.16 | | 21.22 | 32.45 |
| 0.013 | 3.18 | | 21.35 | 29.94 |
| 0.020 | 3.00 | | 20.14 | 48.83 |
| 0.014 | 2.80 | | 18.80 | 36.62 |
| 0.020 | 2.78 | | 18.67 | 52.70 |
| | | MEAN | 18.89 | 40.53 |
| | | SD | 1.46 | 7.88 |
| | | SEM | 0.30 | 1.61 |
| | | N = | 24 | |

TABLE 10
Group 2: Puff APF Foam

| RAW | RAW CAL- | | DEPTH | FLUORIDE |
|---|---|---|---|---|
| FLUORIDE DATA | CIUM DATA | | MIC | PPM |
| 0.560 | 2.69 | | 18.06 | 1524.87 |
| 0.610 | 2.73 | | 18.33 | 1636.68 |
| 1.050 | 2.78 | | 18.67 | 2766.57 |
| 0.670 | 2.80 | | 18.80 | 1752.73 |
| 0.640 | 3.00 | | 20.14 | 1562.63 |
| 0.710 | 2.76 | | 18.53 | 1884.29 |
| 0.590 | 3.18 | | 21.35 | 1359.01 |
| 0.770 | 2.59 | | 17.39 | 2177.65 |
| 0.560 | 3.10 | | 20.81 | 1323.20 |
| 0.880 | 2.67 | | 17.93 | 2414.18 |
| 0.710 | 2.79 | | 18.73 | 1864.03 |
| 0.590 | 2.43 | | 16.32 | 1778.46 |
| 0.510 | 2.68 | | 17.99 | 1393.91 |
| 0.560 | 2.45 | | 16.45 | 1674.25 |
| 0.590 | 2.41 | | 16.18 | 1793.22 |
| 0.640 | 3.03 | | 20.34 | 1547.16 |
| 0.560 | 2.91 | | 19.54 | 1409.59 |
| 1.050 | 2.60 | | 17.46 | 2958.11 |
| 0.590 | 2.92 | | 19.61 | 1480.02 |
| 0.770 | 2.98 | | 20.01 | 1892.66 |
| 0.710 | 2.83 | | 19.00 | 1837.68 |
| 0.740 | 2.73 | | 18.33 | 1985.49 |
| 0.590 | 2.43 | | 16.32 | 1778.46 |
| 0.710 | 2.62 | | 17.59 | 1984.97 |
| | | MEAN | 18.50 | 1824.16 |
| | | SD | 1.44 | 414.64 |
| | | SEM | 0.29 | 84.64 |
| | | N = | 24 | |

TABLE 11
Group 3: Taste APF Gel

| RAW | RAW CAL- | | DEPTH | FLUORIDE |
|---|---|---|---|---|
| FLUORIDE DATA | CIUM DATA | | MIC | PPM |
| 0.415 | 2.21 | | 14.84 | 1375.48 |
| 0.640 | 2.10 | | 14.10 | 2232.33 |
| 0.610 | 2.14 | | 14.37 | 2087.92 |
| 0.800 | 2.67 | | 17.93 | 2194.71 |
| 0.430 | 2.39 | | 16.05 | 1317.86 |
| 0.450 | 2.25 | | 15.11 | 1464.97 |
| 0.640 | 2.18 | | 14.64 | 2150.41 |
| 0.400 | 1.74 | | 11.68 | 1683.87 |
| 0.430 | 2.42 | | 16.25 | 1301.52 |
| 0.510 | 2.35 | | 15.78 | 1589.64 |
| 0.400 | 1.84 | | 12.35 | 1592.36 |
| 0.400 | 1.97 | | 13.23 | 1487.28 |
| 0.840 | 2.50 | | 16.79 | 2461.14 |
| 0.610 | 2.47 | | 16.58 | 1808.97 |
| 0.590 | 2.04 | | 13.70 | 2118.46 |
| 0.640 | 2.54 | | 17.05 | 1845.63 |
| 0.800 | 2.47 | | 16.58 | 2372.42 |
| 0.495 | 2.61 | | 17.52 | 1389.19 |
| 0.380 | 2.36 | | 15.85 | 1179.42 |
| 0.415 | 2.41 | | 16.18 | 1261.33 |
| 0.470 | 2.50 | | 16.79 | 1377.07 |
| 0.880 | 2.08 | | 13.97 | 3098.97 |
| 0.430 | 2.53 | | 16.99 | 1244.93 |
| 0.670 | 2.16 | | 14.50 | 2272.06 |
| | | MEAN | 15.37 | 1787.83 |
| | | SD | 1.64 | 494.60 |
| | | SEM | 0.34 | 100.96 |
| | | N = | 24 | |

TABLE 12
Group 4: Two Part Rinse From Liquid

| RAW | RAW CAL- | | DEPTH | FLUORIDE |
|---|---|---|---|---|
| FLUORIDE DATA | CIUM DATA | | MIC | PPM |
| 0.212 | 2.56 | | 17.19 | 606.59 |
| 0.330 | 2.74 | | 18.40 | 882.19 |
| 0.188 | 2.48 | | 16.65 | 555.27 |
| 0.230 | 3.00 | | 20.14 | 561.57 |
| 0.195 | 2.58 | | 17.32 | 553.62 |
| 0.203 | 2.70 | | 18.13 | 550.72 |
| 0.290 | 3.06 | | 20.55 | 694.18 |
| 0.163 | 2.55 | | 17.12 | 468.21 |
| 0.245 | 2.82 | | 18.93 | 636.38 |
| 0.245 | 2.66 | | 17.86 | 674.66 |
| 0.195 | 2.24 | | 15.04 | 637.65 |
| 0.212 | 2.41 | | 16.18 | 644.34 |
| 0.230 | 2.50 | | 16.79 | 673.88 |
| 0.265 | 2.48 | | 16.65 | 782.69 |
| 0.245 | 2.73 | | 18.33 | 657.36 |
| 0.203 | 3.11 | | 20.88 | 478.12 |
| 0.315 | 2.98 | | 20.01 | 774.27 |
| 0.315 | 3.21 | | 21.55 | 718.79 |
| 0.290 | 2.96 | | 19.87 | 717.64 |
| 0.290 | 2.76 | | 18.53 | 769.64 |
| 0.245 | 2.81 | | 18.87 | 638.64 |
| 0.212 | 3.08 | | 20.68 | 504.18 |
| 0.203 | 2.61 | | 17.52 | 569.71 |
| 0.170 | 2.41 | | 16.18 | 516.69 |
| | | MEAN | 18.31 | 636.12 |
| | | SD | 1.74 | 105.41 |
| | | SEM | 0.35 | 21.52 |
| | | N = | 24 | |

TABLE 13
Group 5: Two Part Rinse From Powder, Administered Simultaneously

| RAW | RAW CAL- | | DEPTH | FLUORIDE |
|---|---|---|---|---|
| FLUORIDE DATA | CIUM DATA | | MIC | PPM |
| 0.188 | 2.79 | | 18.73 | 493.57 |
| 0.230 | 2.39 | | 16.05 | 704.90 |
| 0.170 | 2.62 | | 17.59 | 475.28 |
| 0.180 | 2.71 | | 18.20 | 486.52 |
| 0.143 | 2.82 | | 18.93 | 371.44 |
| 0.345 | 2.89 | | 19.40 | 874.42 |
| 0.315 | 3.12 | | 20.95 | 739.53 |
| 0.315 | 2.91 | | 19.54 | 792.89 |
| 0.245 | 2.66 | | 17.86 | 674.66 |
| 0.290 | 2.46 | | 16.52 | 863.50 |
| 0.180 | 2.66 | | 17.86 | 495.67 |
| 0.163 | 2.63 | | 17.66 | 453.97 |
| 0.195 | 2.74 | | 18.40 | 521.29 |
| 0.203 | 2.76 | | 18.53 | 538.75 |
| 0.163 | 2.69 | | 18.06 | 443.85 |
| 0.180 | 2.82 | | 18.93 | 467.54 |
| 0.195 | 2.69 | | 18.06 | 530.98 |
| 0.230 | 3.15 | | 21.15 | 534.83 |
| 0.188 | 2.81 | | 18.87 | 490.06 |
| 0.212 | 2.83 | | 19.00 | 548.72 |
| 0.170 | 2.93 | | 19.67 | 424.99 |
| 0.230 | 2.76 | | 18.53 | 610.40 |
| 0.222 | 2.56 | | 17.19 | 635.20 |
| 0.280 | 2.73 | | 18.33 | 751.27 |
| | | MEAN | 18.50 | 580.18 |
| | | SD | 1.17 | 141.92 |
| | | SEM | 0.24 | 28.97 |
| | | N = | 24 | |

TABLE 14
Group 6: Two Part Rinse From Powder, Administered Sequentially

| RAW | RAW CAL- | | DEPTH | FLUORIDE |
|---|---|---|---|---|
| FLUORIDE DATA | CIUM DATA | | MIC | PPM |
| 0.960 | 2.51 | | 16.85 | 2801.53 |
| 1.230 | 2.39 | | 16.05 | 3769.68 |
| 0.670 | 2.51 | | 16.85 | 1955.23 |
| 0.840 | 2.51 | | 16.85 | 2451.34 |
| 1.130 | 2.66 | | 17.86 | 3111.68 |
| 1.000 | 2.60 | | 17.46 | 2817.24 |
| 0.590 | 2.03 | | 13.63 | 2128.89 |
| 1.000 | 2.65 | | 17.79 | 2764.09 |
| 0.740 | 2.30 | | 15.44 | 2356.69 |
| 0.800 | 2.32 | | 15.58 | 2525.80 |
| 0.960 | 2.35 | | 15.78 | 2992.27 |
| 0.840 | 2.21 | | 14.84 | 2784.10 |
| 1.230 | 2.43 | | 16.32 | 3707.63 |
| 1.000 | 2.46 | | 16.52 | 2977.57 |
| 0.880 | 2.61 | | 17.52 | 2469.68 |
| 1.130 | 3.01 | | 20.21 | 2749.85 |
| 0.740 | 2.81 | | 18.87 | 1928.96 |
| 0.740 | 2.45 | | 16.45 | 2212.40 |
| 1.000 | 2.92 | | 19.61 | 2508.50 |
| 0.740 | 2.54 | | 17.05 | 2134.01 |
| 0.800 | 2.70 | | 18.13 | 2170.32 |
| 1.230 | 2.58 | | 17.32 | 3492.07 |
| 0.610 | 2.43 | | 16.32 | 1838.74 |
| 1.050 | 2.59 | | 17.39 | 2969.53 |
| | | MEAN | 16.95 | 2650.74 |
| | | SD | 1.45 | 532.69 |
| | | SEM | 0.30 | 108.74 |
| | | N = | 24 | |

From the foregoing, it will be appreciated that, although specific embodiments of the invention have been described herein for purposes of illustration, various modifications may be made without deviating from the spirit and scope of the invention. Accordingly, the invention includes all permutations and combinations of the subject matter set forth herein and is not limited except as by the appended claims.

What is claimed is:

1. A dental fluoride rinse system comprising:
a) a first dry dental rinse composition comprising an acidifier, a fluoride ion source other than stannous fluoride able to provide a fluoride ion when mixed with water, an effective amount of a phosphate ion source, and a pharmaceutically acceptable carrier or diluent, wherein when the first dry dental rinse composition is mixed with a desired amount of water the resulting solution is about pH 3.75±0.75 and comprises greater than about 1% by weight of the fluoride ion; and,
b) a second dry dental rinse composition comprising stannous fluoride and a pharmaceutically acceptable carrier or diluent.

2. The dental fluoride rinse system of claim 1 wherein when the second dry dental rinse composition is mixed with a desired amount of water the resulting solution comprises between about 0. 1% and 1% stannous fluoride.

3. The dental fluoride rinse system of claim 1 wherein the first and second compositions further comprise a desired flavoring.

4. The dental fluoride rinse system of claim 1 wherein the first rinse composition further comprises a buffer.

5. The dental fluoride rinse system of claim 1 wherein the acidifier of the first rinse composition comprises an acidic buffer.

6. The dental fluoride rinse system of claim 1 wherein the first and second compositions further comprise a sweetener.

7. The dental fluoride rinse system of claim 1 wherein the fluoride ion source of the first rinse composition is selected from the group consisting of sodium fluoride, lithium fluoride, sodium monofluorophosphate, salts of fluorosilic acid, sodium fluoborate, sodium bifluoride, potassium bifluoride, strontium fluoride, indium fluoride, zirconium fluoride, ammonium fluoride, amine fluoride, titanium tetra fluoride, sodium hexafluorostannate, sodium fluorozirconate, potassium fluorozirconate, ammonium fluorozirconate, copper fluoride, nickel fluoride, palladium fluoride, manganese fluoride, stannous chlorofluoride and stannous fluorozirconate.

8. The dental fluoride rinse system of claim 7 wherein the fluoride ion source is an alkali metal fluoride selected from the group consisting of sodium fluoride, potassium fluoride and mixtures thereof.

9. The dental fluoride rinse system of claim 1 wherein the acidifier is selected from the group consisting of citric acid, lactic acid, malic acid, and tartaric acid.

10. The dental fluoride rinse system of claim 1 wherein the pharmaceutically acceptable carrier or diluent is selected from the group consisting of sorbitol, xylitol, tartaric acid, salts of citric acid, salts of lactic acid, block copolymers of ethylene oxide and propylene oxide nonionic surfactants, sodium acetate, sodium bicarbonate and sodium chloride.

11. The dental fluoride rinse system of claim 1 wherein the phosphate ion source is selected from the group consisting of monobasic sodium phosphate, dibasic sodium phosphate, tribasic sodium phosphate, sodium pyrophosphate, and potassium pyrophosphate.

12. The dental fluoride rinse system of claim 1 wherein the first rinse composition and the second rinse composition are contained in separate vessels.

13. The dental fluoride rinse system of claim 1 wherein the separate vessels are foil pouches.

14. The dental fluoride rinse system of claim 1 wherein the system further comprises a graduated mixing cup sized to receive and measure a predetermined amount of a pharmaceutically acceptable solvent between about 10 ml and 20 ml and to permit mixing of at least one of the first and second rinse compositions with the solvent.

15. The dental fluoride rinse system of claim 1 wherein the first rinse composition and the second rinse composition are powders.

16. A dental fluoride rinse system comprising:
a) a first dry dental rinse composition comprising a means for acidifying, a means for providing a fluoride ion other than stannous fluoride, means for providing a phosphate ion, a pharmaceutically acceptable carrier or diluent means, and a buffer, wherein when the first dry dental rinse composition is mixed with water the resulting solution is about pH 3.75±0.75 and comprises greater than about 1% by weight of the fluoride ion; and,
b) a second dry dental rinse composition comprising a means for providing a stannous fluoride and a pharmaceutically acceptable carrier or diluent means, wherein when the second dry dental rinse composition is mixed with a desired amount of water the resulting solution comprises between about 0.1% and 1% stannous fluoride.

17. The dental fluoride rinse system of claim 16 wherein the first and second compositions further comprise at least one of a means for providing a desired flavor, a buffer means, and a means for providing artificial sweetening.

18. A method of preparing a dental fluoride rinse comprising:
a) mixing a first solvent with a first dry dental rinse composition comprising an acidifier, a fluoride ion source other than stannous fluoride able to provide fluoride ion when mixed with water, a phosphate ion source and a pharmaceutically acceptable carrier or diluent, to provide a first dental rinse of about pH 3.75±0.75 and comprising greater than about 1% by weight of the fluoride ion; and
b) mixing a second solvent with a second dry dental rinse composition comprising stannous fluoride and a pharmaceutically acceptable carrier or diluent to provide a second dental rinse comprising between about 0.1% and 1% stannous fluoride.

19. The method of claim 18 wherein the second solvent comprises water.

20. The method of claim 18 wherein the first and second dry dental rinse compositions each further comprise at least one of a desired flavoring and an artificial sweetener.

21. A method of administering a dental fluoride rinse to a patient comprising:
a) mixing water with a first dry dental rinse composition comprising an acidifier, a fluoride ion source other than stannous fluoride able to provide fluoride ion when mixed with water, a phosphate ion source and a pharmaceutically acceptable carrier or diluent, to provide a first dental rinse of about pH 3.75±0.75 and comprising greater than about 1% by weight of the fluoride ion;
b) mixing water with a second dry dental rinse composition comprising stannous fluoride and a pharmaceutically acceptable carrier or diluent to provide a second dental rinse comprising between about 0.1% and 1% stannous fluoride; and,
c) serially administering the first dental rinse and then the second dental rinse to a patient.

22. The method of claim 21 wherein the first and second dry dental rinse compositions a administered to a patient within about 20 minutes of the mixing.

23. The method of claim 21 wherein the first and second dry dental rinse compositions each further comprise at least one of a desired flavoring and an artificial sweetener.

24. A kit comprising a first vessel containing a first dry dental rinse composition according to claim 1 and a second vessel containing a second dry dental rinse composition according to claim 1.

25. The kit of claim 24 wherein the first and second vessels are foil pouches.

26. A kit comprising a first means for containing a first dry dental rinse composition according to claim 16 and a second means for containing a second dry dental rinse composition according to claim 16.
